(19) United States
(12) Patent Application Publication
(10) Pub. No.: US 2013/0130236 A1
(43) Pub. Date: May 23, 2013

(54) SEPARATION METHOD OF LABELED CELLS AND USES THEREOF

(71) Applicants: Jui-I Chao, Hsinchu City (TW); Zhi-Yi Lien, Hsinchu City (TW); Kuo-Chu Hwang, Hsinchu City (TW)

(72) Inventors: Jui-I Chao, Hsinchu City (TW); Zhi-Yi Lien, Hsinchu City (TW); Kuo-Chu Hwang, Hsinchu City (TW)

(73) Assignee: NATIONAL CHIAO TUNG UNIVERSITY, Hsinchu City (TW)

(21) Appl. No.: 13/471,768

(22) Filed: May 15, 2012

(30) Foreign Application Priority Data
Nov. 17, 2011 (TW) 100141978

(51) Int. Cl. G01N 21/64; C12N 5/071; C12Q 1/02; G01N 27/26; C12N 13/00; C12N 5/09; B82Y 15/00; B82Y 25/00

(52) U.S. Cl. 435/6.1; 435/173.9; 435/325; 435/29; 435/34; 204/461; 977/834; 977/904

(57) ABSTRACT

A method for separating labeled cells and a use (of the labeled cells) thereof are provided. More specifically, a method for labeling cells using fluorescent magnetic nanodiamonds, and a method for separating the labeled cells using the labeling method by the fluorescent or magnetic properties of the nanodiamonds are provided.

### BACKGROUND OF THE INVENTION

1. Field of the Invention

The present invention relates to separation methods and uses thereof, and more particularly, relates to a separation method for fluorescent magnetic nanodiamond-labeled cells and a use thereof.

2. Description of Related Art

In recent years, a variety of functional nanomaterials have been developed for applications in the biomedical field, such as the use of nanosomes for carrying drugs in cancer therapies (Batist G. et al., J. Clin Oncol. 2001, March 1; 19(5):1444-54; Chen Y., et al., Mol. Ther. 210 April; 18(4):828-34); the use of a fluorescent-bound nanoscale magnet to perform dynamic tracking and cell labeling (Maxwell D. J., et al., Stem Cells. 2008 February; 26(2):517-24; Ruan J. et al., Int J Nanomedicine, 20116:425-35.); the use of a combination of nanoscale quantum dots and silicon oxide in biological imaging (Erogbogbo F., et al., ACS Nano. 2011 Jan. 25; 5(1):413-23); the use of a combination of nanoscale quantum rods and phospholipid in tumor imaging and tacking (Yong K. T., et al., ACS Appl Mater Interfaces. 2009 March; 1(3):710-9); and the use of nanotubes for carrying drugs in cancer therapies (Liu Z. et al., Cancer Res. 2008 Aug. 15; 68(16):6652-60).

The limitations of the applications of nanomaterials in the biomedical field include the difficulty to track the positions and movements of the nanomaterials in vivo, the toxicity of quantum dots to organisms and the uncertain biocompatibility of nanoscale carbon materials such as carbon nanotubes and fullerenes.

As substitutes for the aforesaid nanomaterials, a nanodiamond (ND) is a nanomaterial applicable in the biomedical field, due to its excellent biocompatibility and low oxidative pressure induction (i.e., lower toxicity) as compared with other nanoscale carbon materials. It has been corroborated that ND can be used in many cell lines without obvious cytotoxicity (Liu K. K., et al., Biophys J. 2007 Sep. 15; 93(6):2199-208; Yu S. J., et al., J Am Chem. Soc. 2005 Dec. 21; 127(50):17604-5; Vaijayanthimala V., et al., Nanotechnology. 2009 Oct. 21; 20(42):425103; Huang H., et al., Nano Lett. 2007 November; 7(11):3305-14; Schrand A. M., et al., Nanoscale. 2011 February; 3(2):435-45). At the same time, ND does not lead to obvious abnormality in cell division, differentiation and morphological changes in embryonic development (Liu K. K., et al., Biomaterials 2009; 30(26):249-59; Mohan N., et al., Nano Lett. 2010; 10(9):3692-3699).

In terms of applications in the biomedical field, a ND has many advantages due to its biocompatibility, electrochemical and optical properties. (For example, biological molecules or therapeutic agents can be provided, either by chemical modification of the ND, or by allowing physical attachment to the ND, which acts as a convenient binding platform for chemical agents). The NDs with functionalized surface can bind to fluorescent molecules (Schrand A. M., et al., Nanoscale 2011; 3(2):435-445; Chang I. P., et al., J Am Chem Soc 2008; 130(46):15476-15481); lysozyme (Chao J. I., et al., Biophys J 2007; 93(6):2199-2208); growth hormones (Cheng C. Y., et al., Appl Phys Lett 2007; 90(16):3); DNA (Zhang X. Q., et al., ACS Nano 2009; 3(9):2609-2616); cytochrome c (Huang L. C., et al., Langmuir 2004; 20(14):5876-5884); proteins (Hartl A., et al., Nat Mater 2004; 3(10);736-742); and anti-cancer agents (Huang H., et al., Nano Lett 2007; 7(11):3305-3314; Liu K. K., et al., Nanotechnology 2010; 21(31):14); A ND can emit bright fluorescence, and does not bring about photobleaching and light scintillation (Yu S. J., et al., J Am chem. Soc 2005; 127(50): 17604-17605; Chao J. I., et al., Biophys J 2007; 93(6):2199-2208). The fluorescent property can be introduced to a ND by chemically modifying the ND with a fluorescent molecule, or by binding a fluorescent molecule to the ND (Schrand A. M., et al., Nanoscale 2010; 3(2):435-445; Chang I. P., et al., J Am Chem Soc 2008; 130(46):15476-15481; Hens S. C., et al., Diamond Relat Mater 2008; 17(11):1858-1866). In addition to the fluorescent property, a ND with magnetic property can be used in magnetic resonance imaging (MRI) detection. It has been reported that ion implantation of isotopes (¹⁵N) and (¹²C) can create magnetism in ND particles by generating sp²/sp³ defects (Talapatra S, et al., Phys Rev Lett 2005; 95(9):097201). Further, magnetic nanoparticles can be fixed onto the surfaces of NDs by using a metal-containing compound, to thermally damage a hot mixture of mineral oil and ND (Gubin S. P., et al., Diamond Relat Mater 2007; 16(11):1924-1928).

It has been reported that by using a microwave-arcing process, iron nanoparticles (ferrocence) is chemically bonded to the surface of an ND via a grapheme layer, to form a ND with fluorescence and magnetism (so-called fluorescent magnetic nanodiamond (FMND)) (Chang I. P., et al., J Am Chem Soc 2008; 130(46):15476-18481). Magnetic nanodiamond (MND) is chemically modified by surface grafting using polyacrylic acid, so as to allow the fluorescent portion to be covalently bonded to the surface of MND, and thereby converting MND to FMND. The fluorescent portion used in FMND is fluorescein O-methylacrylate, which emits bright green fluorescence at a wavelength of 488 nm, and collects the emitted light at a wavelength of 510 nm to 530 nm.

Although a theory postulating that endocytic ND clusters can be segregated during cell division and remain as a single ND cluster, there is currently no report on methods for separating ND-labeled cells and whether such labeled cell lines survive or have the ability to be sub-cultured.

### SUMMARY OF THE INVENTION

The present invention provides a method for separating labeled cells and a use of the labeled cells. More specifically, the present invention relates to a method for separating cells labeled with FMNDs and a use thereof.

In an aspect of the present invention, a method for separating labeled cells is provided, wherein the method comprises the following steps: culturing target cells; co-culturing the target cells with the solution, so as to allow the FMNDs in the solution to label the target cells; and separating the FMND-labeled target cells from the co-culture of target cells and solution by means of the fluorescence and magnetism of the labeled target cells.

Further, the FMND-labeled target cells are separated by means of the difference between the fluorescence intensity of the FMND-labeled target cells and that of the unlabeled cells. In an embodiment, a flow cytometer is used to screen the FMND-labeled target cells.

Preferably, the method further comprises steps of washing of the co-culture of target cells and the FMND solution prior to use of the flow cytometer, so as to collect the FMND-labeled target cells and the unlabeled target cells; the method also comprises the subsequent concentration of the FMND-labeled target cells and the unlabeled target cells by centrifugation.

In another embodiment, a magnetic device is used to separate the FMND-labeled target cells. Preferably, the method further comprises the steps of washing of the co-culture of target cells and FMND solution prior to the use of a magnetic device, so as to collect the FMND-labeled target cells and the unlabeled target cells; the method further comprises concentrating the FMND-labeled target cells and the unlabeled target cells by centrifugation; and suspending the FMND-labeled target cells and the unlabeled target cells in a container with a buffer solution, so as to allow the magnetic device to adhere the FMND-labeled target cells onto the tube walls of the container.

The separation method of the present invention further comprises the step of cryopreserving the sorted FMND-labeled target cells.

In yet another aspect of the present invention, a separated cell is provided, wherein the cell is one that is separated according to the separation method of the present invention.

Further, the labeled and separated cell is used for standardization, detection, imaging and tracking of cells, the analysis of biomolecular activity and the screening of drug activity.

Further, the cell is an animal cell, including a cancer cell and a stem cell.

The present invention uses FMND-labeled target cells, and utilizes the fluorescence and magnetism of the FMND to separate the cells labeled by the labeling method. The labeled cells can continue to survive, be stored and be further cultured. The labeled cells are suitable for applications such as standardization, detection, imaging and tracking of cells, analysis of biomolecular activity and screening of drug activity.

### BRIEF DESCRIPTIONS OF THE DRAWINGS

FIG. 1 is a graph showing the size distribution of FMNDs in distilled deionized water (DDW) and PBS.

FIG. 2 is a graph showing the effects of FMND on cell viability of HFL-1 normal lung fibroblasts.

FIG. 3 is a graph showing the effects of FMND on cell viability of A549 lung cancer cells.

FIG. 4 is a graph showing the effects of FMND on cell growth ability of A549 lung cancer cells.

FIG. 5 is a set of microscopic images of FMND in A549 cells, as detected under a laser scanning confocal microscope.

FIG. 6 is a graph showing the fluorescence intensity of FMND in A549 cells.

FIG. 7 is a graph showing the particle complexity of FMND in A549 cells.

FIG. 8 is a graph showing the cell size distribution of A549 cells after being treated with different concentrations of FMND particles.

FIGS. 9A and 9B are graphs showing the separations of FMND-bearing cells by a flow cytometer with a sieve.

FIG. 10 shows a set of images obtained by performing fluorescent imaging on the FMND-bearing cells separated by the flow cytometer with a sieve.

FIG. 11 shows a set of images comparing the morphology and viability of parent cells and the FMND-bearing cells.

FIG. 12 shows an electrophoregram comparing total protein expression profiles of the parent cells and the FMND-bearing cells.

FIG. 13 is a graph showing the fluorescence intensity detected in the FMND-bearing cells separated by the flow cytometer.

FIG. 14 is a set of images showing cell morphology and viability of different generations of cells bearing FMND separated by a magnetic device.

FIG. 15 is an electrophoregram comparing the total protein expression profiles of the parent cells and the different generations of cells bearing FMND.

FIG. 16 is a graph showing the fluorescence intensity detected in different generations of cells bearing FMND.

FIG. 17 is a set of images showing the fluorescence intensity detected in FMND-bearing cells under a laser scanning confocal microscope.

FIG. 18 is a graph showing the separating ratios of the different generations of cells bearing FMND.

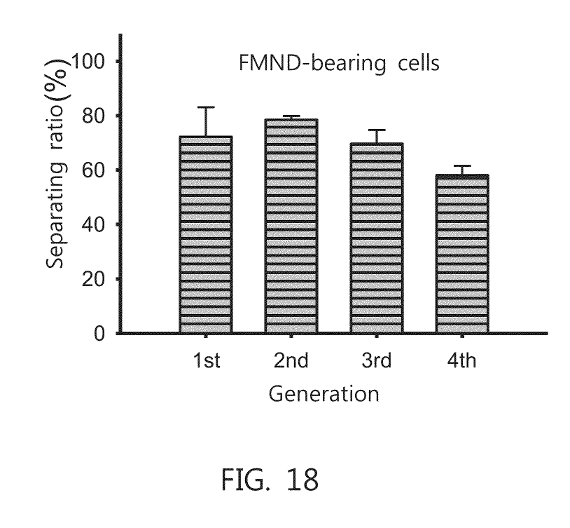

FIG. 19 is a set of images comparing the cell morphology and viability of the parent cells and the re-dissolved FMND-bearing cells.

FIG. 20 is a set of images showing separation and identification of various FMND-bearing cancer cell types.

### DETAILED DESCRIPTION OF THE PREFERRED EMBODIMENTS

The present invention is further exemplified by the following examples, but the examples should not be construed as intending to limit the scope of the present invention.

### Preparation of Fluorescent Magnetic Nanodiamond (FMND)

FMNDs were prepared as described using a published method (Chang I. P., et al., Am Chem Soc 2008; 130(46):15476-18481). Specifically, magnetic nanodiamonds (MNDs) were composed of pristine NDs and iron nanoparticles (ferrocene) via a microwave-arcing process. The ferrocene particles and NDs formed MNDs by chemically bonding. To introduce fluorescence in MNDs, MNDs were converted into FMNDs by covalent surface grafting with polyacrylic acids and fluorescein o-methacrylate. FMND particles were dissolved in distilled de-ionized water or phosphate-buffered saline (PBS), before the treatment of cells using FMND.

### Dynamic Light Scattering (DLS) Analysis

To examine the size distribution of FMNDs dissolved in DDW and PBS, the concentration of FMND particles in DDW or PBS (0.5 mg/ml) was prepared and analyzed by DLS (BI-200SM, Brookhaven Instruments Co., Holtsville, N.Y.). In a particular suspension, when a beam of laser hits the particle, the particles scattered some of the laser. The measured data were subjected to the BIC dynamic light scattering software (Brookhaven Instruments Co.). The scattered light changed over time, and the average particle size was calculated by the variation of scattered light.

### Cell Culture

HFL-1 cells (ATCC #CCL-153) were normal lung fibroblasts derived from a Caucasian fetus. The A549 lung epithelial cell line (ATCC #CCL-185) was derived from the lung adenocarcinoma of a 58 year-old Caucasian male. RKO was a human colon carcinoma cell line. BFTC905 cells were derived from human bladder carcinoma. MCF-7 was a human breast cancer cell line. HeLa was a c human cervical cancer cell line. HFL-1 and HeLa cells were maintained in DMEM medium (Invitrogen Co., Carlsbad, Calif.). A549, BFTC905 and MCF-7 cells were maintained in RPMI-1640 medium (Invitrogen). The complete media contained 10% fetal bovine serum (FBS), 100 unit/ml penicillin and 100 mg/ml streptomycin. These cells were incubated at 37° C., and maintained in 5% CO₂ in a humidified incubator (310/Thermo, Form a Scientific, Inc., Marietta, Ohio).

### MTT Assays

The cells were plated in 96-well plates at a density of 1×10⁴ cells/well for 16 to 20 hours. The cells were treated with or without FMNDs for 24 hours in complete medium. Subsequently, the medium was replaced and the cells were incubated with 0.5 mg/ml of 3-(4,5-dimethyl-thiazol-2-yl)-2,5-diphenyl tetrazolium bromide (MTT) (Sigma Chemical Co., St. Louis, Mo.) in complete medium for 4 hours. The survived cells converted MTT to formazan, which generated a blue-purple color when dissolved in dimethyl sulfoxide (DMSO). The intensity of formazan was measured at a wavelength of 565 nm using a plate reader (VERSAmax, Molecular Dynamics Inc., CA) for enzyme-linked immunosorbent (ELISA) assays. Cell viability was calculated by dividing the absorbance of the cells treated with FMNDs by that of the cells not treated with FMNDs.

### Cell Growth Assays

A549 cells were seeded at a density of 1×10⁶ cells per 100-mm Petri dish in complete medium for 24 hours. Then, the cells were treated with or without FMNDs (50 μg/ml) for 24 hours. Subsequently, the cells treated or untreated with FMNDs were re-cultured in fresh medium for counting the total cell number every 2 days, for a total of 10 days.

### Cellular Fluorescence Intensity, Particle Complexity and Size Distribution by FMNDs

A549 cells were plated at a density of 7×10⁵ cells per 60-mm Petri dish in complete medium for 16 to 20 hours. After treatment in medium with or without 10 to 100 μg/ml FMNDs, the cells were washed twice with PBS. The cells were trypsinized and collected by centrifugation at 1500 rpm for 5 minutes. The cell pellets were re-suspended in PBS. To avoid cell aggregation, the cell suspension was filtered through a nylon mesh membrane. Finally, the samples were analyzed by a flow cytometer (FACSCalibur, Becton-Dickinson, San Jose, Calif.). A minimum of 10,000 cells were analyzed. The fluorescence of FMNDs was triggered and emitted at a wavelength of 488 nm, and was collected in the green light signal range. The fluorescence intensity, particle complexity and cell size were quantified using a minimum of 10,000 cells by CellQuest software (BD, Biosciences).

### Immunofluorescence Staining and Confocal Microscopy

The cells were cultured on cover slips, and kept in a 35-mm Petri dish for 16 to 20 hours before treatment. After treatment with or without FMNDs, the cells were washed with isotonic PBS (pH 7.4), and then were fixed with 4% paraformaldehyde solution in PBS for 1 hour at 37° C. Thereafter, the cover slips were washed three times with PBS, and non-specific sites were blocked in PBS containing 10% FBS and 0.3% Triton X-100 for 1 hour. The β-tubulin and nuclei were stained with anti-β-tubulin Cy3 (1:100) and Hoechst 33258 (Sigma Chemical co., St. Louis, Mo.) for 30 minutes at 37° C., respectively. Thereafter, the samples were examined under an OLYMPUS confocal microscope (FV500, OLYMPUS, Japan) that was equipped with an UV laser (405 nm), an Ar laser (488 nm) and a HeNe laser (543 nm).

### Separation of FMND-Bearing Cells by a Flow Cytometer

The cells were plated at a density of 2×10⁶ cells per 100-mm Petri dish in complete medium for 24 hours. After treatment with FMNDs (50 μg/ml) for 24 hours, the cells were washed twice with PBS. The washed cells (including the FMND-labeled A549 cells and unlabeled A549 cells) were trypsinized, and collected by centrifugation at 1500 rpm for 5 minutes to collect cell pellets. The cell pellets were re-suspended in 1 to 2 ml ice-cold sorting buffer, which contained 1 mM EDTA, 25 mM HEPES and 2% FBS in PBS. To avoid cell aggregation, the cell suspensions were filtered through a nylon mesh membrane. The fluorescence-activated cell sorting analyses were performed with a FACSCalibur sieve (Becton-Dickinson). The FMDN-bearing cells, which displayed green fluorescence intensity in a flow cytometer, were selected for separation. The separated cells were collected in a 50 ml centrifuge tube that had been coated with 10% FBS on the wall and contained 15 to 20 ml of complete medium inside. After separation, the cell suspensions were centrifuged at 1000 rpm for 10 minutes. Then, the cell pellets were re-suspended in complete medium. The cells were incubated at 37° C., in 5% CO₂ in a humidified incubator, or added 10% DMSO for storage in liquid nitrogen.

### Separation of FMND-Bearing Cells by a Magnetic Device

The cells were plated at a density of 2×10⁶ cells per 100-mm Petri dish in complete medium for 24 hours. After treatment with 50 μg/ml FMNDs for 24 hours, the cells were washed twice with PBS. The washed cells (including the FMND-labeled A549 cells and unlabeled A549 cells) were trypsinized, and collected by centrifugation at 1500 rpm for 5 minutes to collect cell pellets. The cell pellets were re-suspended in 1 ml PBS and transferred to 1.5 ml eppendorf tubes. The eppendorf tubes were placed onto a magnetic rack (Magna GrIP Rack, Millipore, Bedford, Mass.) for at least 3 minutes, until the cell pellets were absorbed on to the tube walls (Think OK, check). Then, the suspensions were removed, and the cell pellets were dissolved in complete medium. The cell suspensions within the eppendorf tubes were repeatedly placed onto the magnetic rack 5 times. Finally, the FMND-bearing cells were incubated at 37° C., in 5% CO₂ in a humidified incubator, or added 10% DMSO for storage in liquid nitrogen.

### SDS-PAGE

To compare the total protein expression profiles between the parental and FMND-bearing cells, the cells were subjected to sodium dodecyl sulfate polyacrylamide gel electrophoresis (SDS-PAGE) analysis. The separated FMND-bearing cells were lysed in the ice-cold cell extraction buffer (pH 7.6), which contained 0.5 mM DTT, 0.2 mM EDTA, 20 mM HEPES, 2.5 mM MgCl₂, 75 mM NaCl, 0.1 mM Na₃VO₄, 50 mM NaF and 0.1% Trinton-X10. The protease inhibitors included 1 μg/ml aprotinin, 0.5 μg/ml leupeptin and 100 μg/ml 4-(2-aminoethyl) benzenesulfonylfluoride were added to the cell suspension. The cell extracts were gently rotated at 4° C. for 30 minutes. After centrifugation, the pellets were discarded, and the protein concentrations of the supernatants were determined by a BCA protein assay kit (Pierce, Rockford, Ill.). Equal amounts of proteins (40 μg) were subjected to electrophoresis by 12% SDS-PAGE. After electrophoresis, the gel was stained with a coomassie blue buffer (0.1% coomassie blue, 10% acetic acid and 45% methanol) for 1 hour.

### Statistical Analysis

Data were analyzed using Student's t test, and a p value of <0.05 was considered as statistically significant in the experiments.

### Size Distribution of FMNDs in DDW and PBS

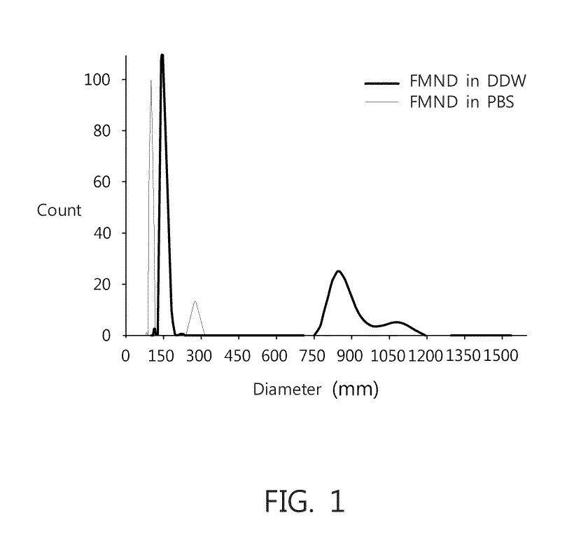

To measure the size distribution of FMNDs, 0.5 mg/ml of FMND solution prepared in DDW or PBS was analyzed by DLS. As shown in FIG. 1, the bold line represents FMNDs dissolved in DDW. The first peak of FMND in DDW was from 137.24 to 176.71 nm, and the second peak was from 805.15 to 1127.86 nm FMND in DDW was 277.7 nm. The thin line represents FMNDs dissolved in PBS. The first peak of FMND in PBS was from 95.22 to 116.34 nm, and the second peak was from 272.58 to 333.04 nm. The average size of FMND in PBS was 131.7 nm.

### No Cytotoxicity was Induced by FMNDs in HFL-1 Normal Fibroblasts and A549 Lung Cancer Cells

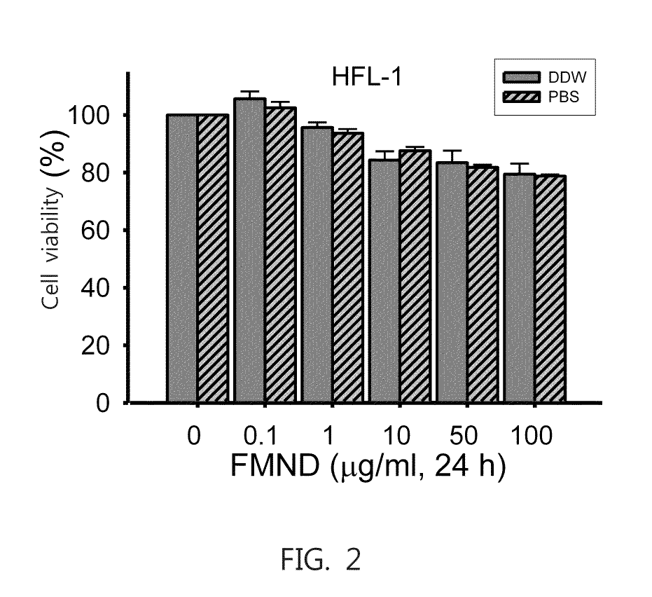

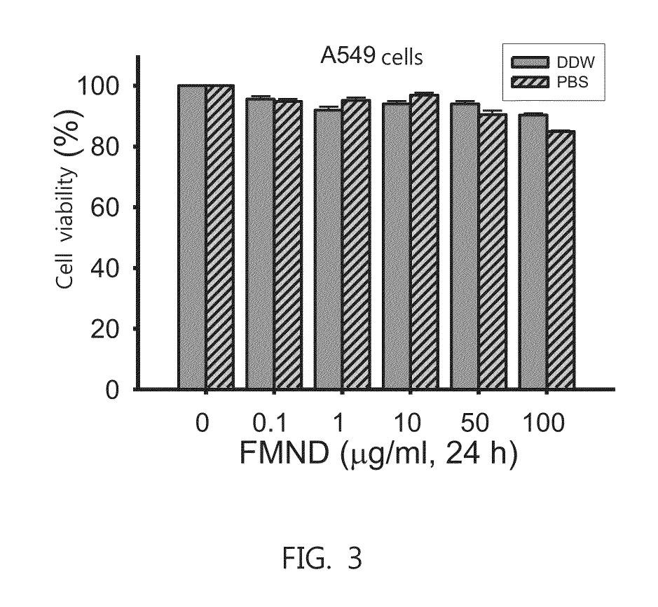

To examine cell cytotoxicity following treatment with FMNDs in human lung cells, HFL-1 normal fibroblasts and A549 human lung carcinoma cells were used. The cells were treated with FMNDs, and analyzed by MTT assays. As shown in FIG. 2, HFL-1 normal fibroblasts treated with FMNDs (0.1 to 100 μg/ml, 24 hours) did not significantly reduce cell viability. As shown in FIG. 3, FMND particles did not induce cytotoxicity in A549 cells.

### No Alteration of the Cell Growth Ability in Lung Cancer Cells

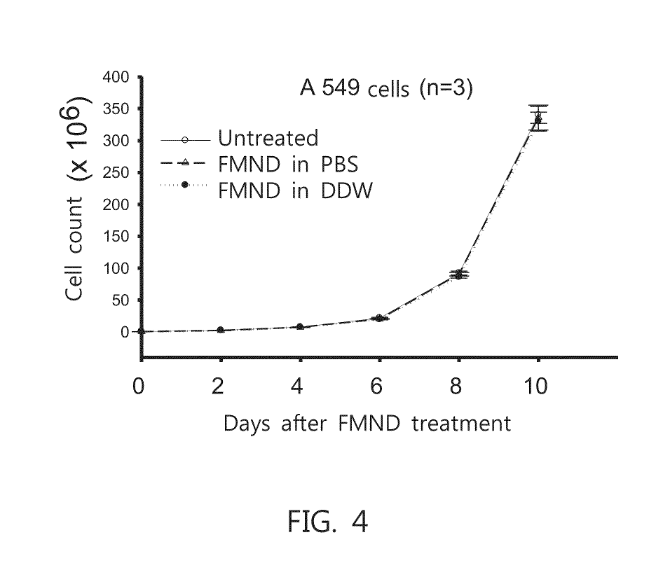

A549 cells were treated with 50 μg/ml of FMND for 24 hours, and then further cultured for another 10 days. The total cell number was analyzed every 2 days. FIG. 4 shows that the FMND particles did not alter the cell growth ability in A549 cells.

### Uptake Ability of FMNDs in Lung Cancer Cells

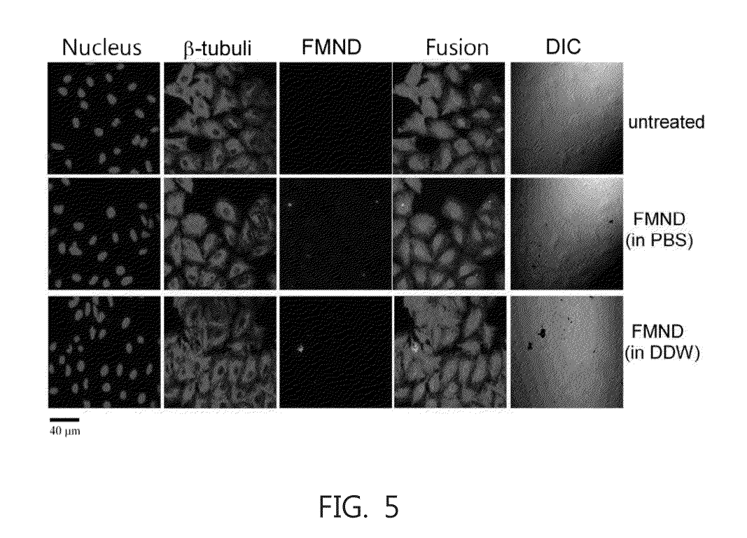

To examine the uptake ability of FMNDs in A549 cells, the cells were treated with FMNDs (50 μg/ml), and analyzed by confocal microscopy. The FMND particles exhibited green fluorescence in A549 cells at a wavelength of 488 nm, and emission was collected in the range of 510 to 530 nm. The cytoskeleton of β-tubulin protein was stained with the Cy3-labeled mouse anti-β-tubulin. The nuclei were stained with Hoechst 33258 that presented with blue color. As shown in FIG. 5, it was observed by differential interference contrast (DIC) that FMND particles did not affect the cell growth ability in A549 cells.

Detection of A549 Lung Cancer Cells with FMNDs by a Flow Cytometer

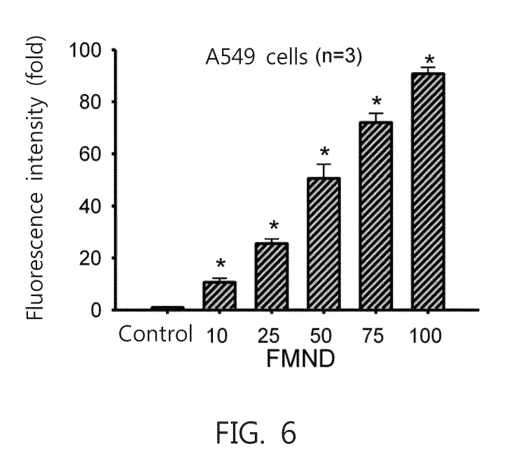

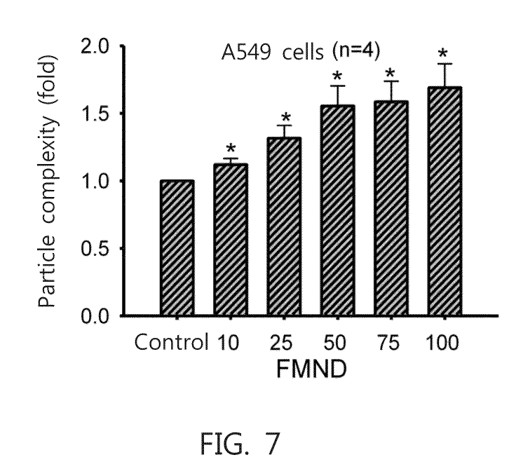

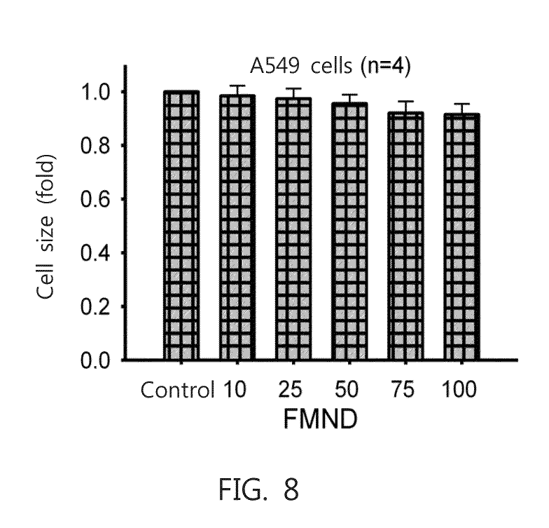

The fluorescence intensity of FMNDs in A549 cells was examined by the flow cytometer. A549 cells were treated with FMNDs (10 to 100 μg/ml, 24 hours), and then analyzed by the flow cytometer. FIG. 6 shows quantitative data indicating that treatment with FMND significantly increased the fluorescence intensity of FMND in A549 cells. The intracellular complexity of FMNDs in A549 cells was examined by SSC-H (lateral scattering light) under the flow cytometer. FIG. 7 shows the quantitative data of FMND treatment (10 to 100 μg/ml, 24 hours), wherein treatment with FMNDs significantly increased the intracellular particle complexity of A549 cells. However, FMNDs did not alter the cell size distribution of FSC-H in A549 cells (FIG. 8).

### Separation of FMND-Bearing Cells by the Flow Cytometer and the Magnetic Device

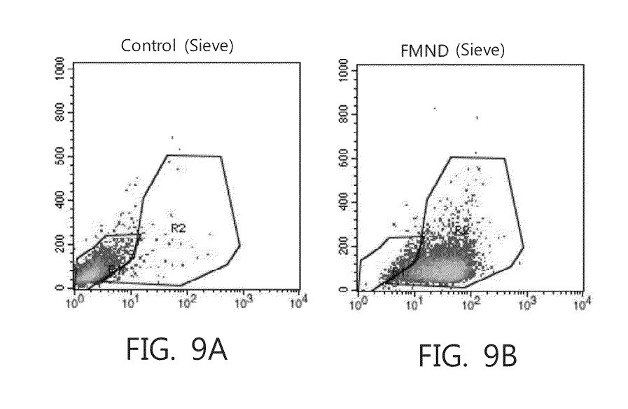

The fluorescent and magnetic properties of FMNDs provided for (contributed to) the separation of ND-bearing cells by FACS-purified function of the flow cytometer with a sieve and magnetic device. To separate the ND-bearing cells, A549 cells were treated with FMND (50 μg/ml, 24 hours), and collected by the flow cytometer with a sieve. FIG. 9A indicates the R1 gate (control, green fluorescence-negative) of the sieve showing the region of parental cells. FIG. 9B indicates the R2 gate showing the region of FMND-bearing cells (fluorescence-activated) and displaying green fluorescent property (Think OK, but Dc). The FMND-bearing cells in the R2 region were collected by the flow cytometer with a sieve.

### Analysis of the FMND-Bearing Cells Separated by the Flow Cytometer

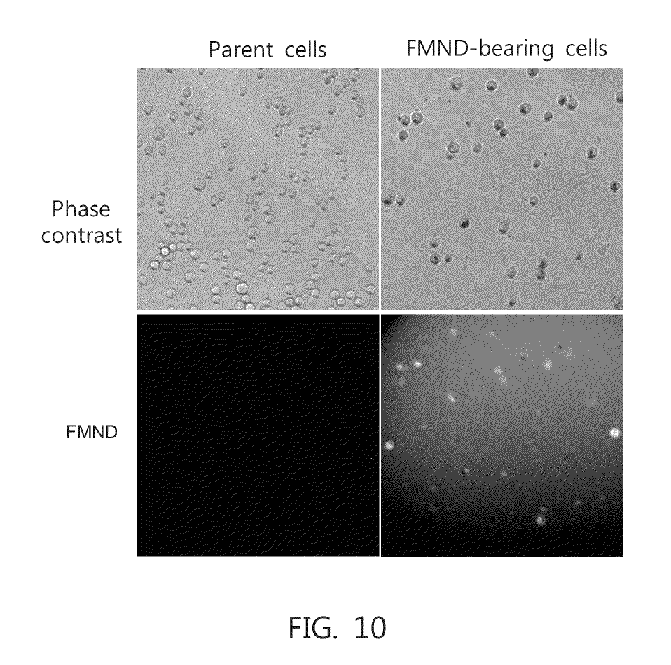

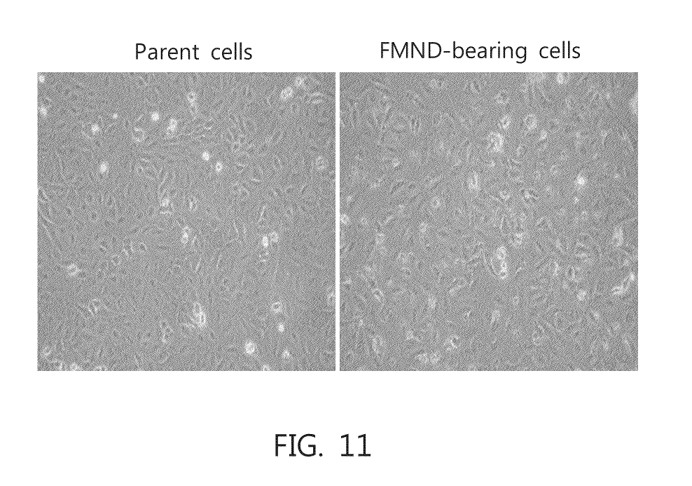

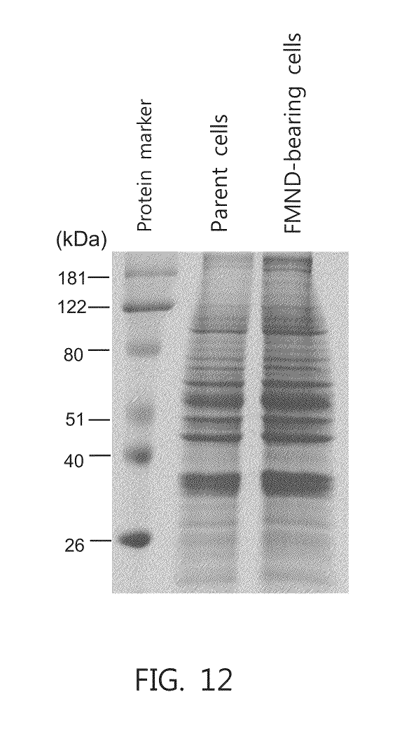

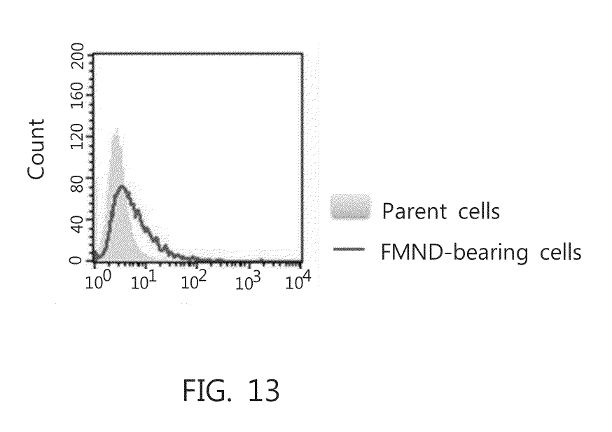

After separation, the FMND-bearing cells were immediately examined under a live-cell imaging system of the fluorescent and phase contrast microscope. Comparison with the parental cells shows that the separated FMND-bearing cells exhibited significant fluorescence intensity under a fluorescent microscope (FIG. 10). Moreover, the FMND-bearing cells were cultured for a further 24 hours. The cell morphology and viability were observed under a phase contrast microscope (FIG. 11). The cell morphology, viability and growth ability of FMND-bearing cells were similar to the parental cells (FIG. 11). Subsequently, the total protein expression profiles of the FMND-bearing cells and the parental cells were examined by the SDS-PAGE analysis. The protein expressions patterns on SDS-PAGE were not significantly altered between the parental cells and the FMND-bearing cells (FIG. 12). Besides, the green fluorescence intensity of the FMND-bearing cells can be detected by the flow cytometer after the cells are cultured for a further 24 hours (FIG. 13).

### Analysis of the FMND-Bearing Cells Separated by the Magnetic Device

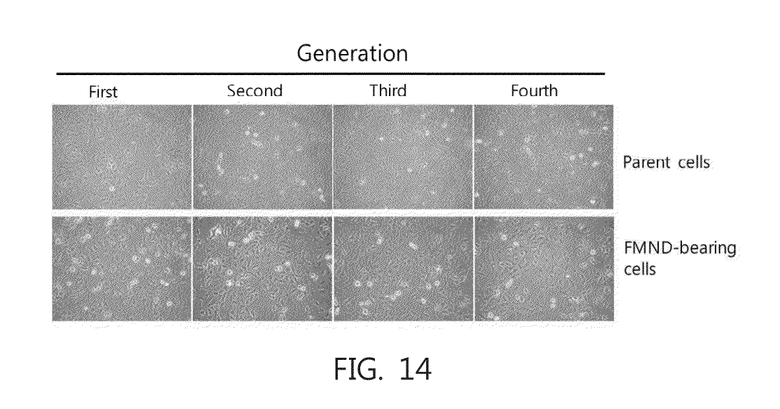

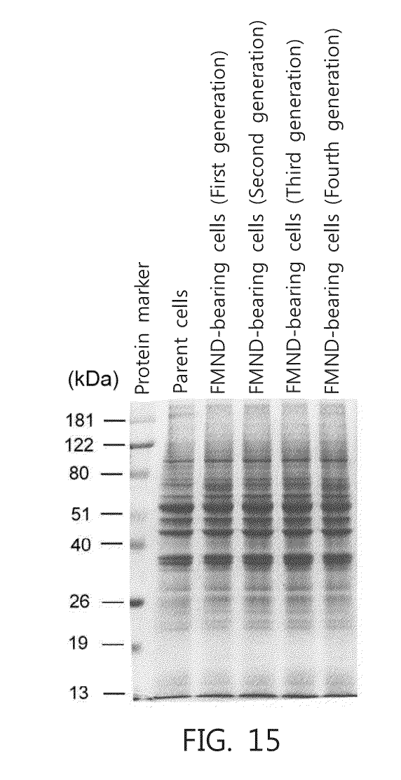

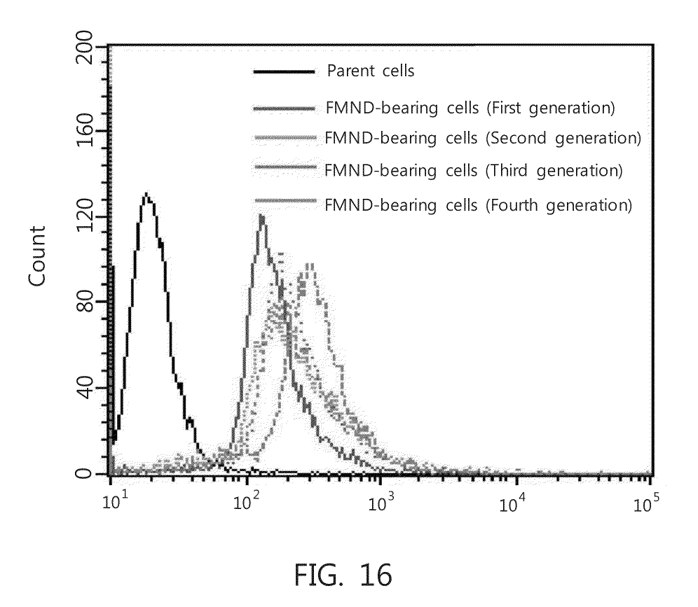

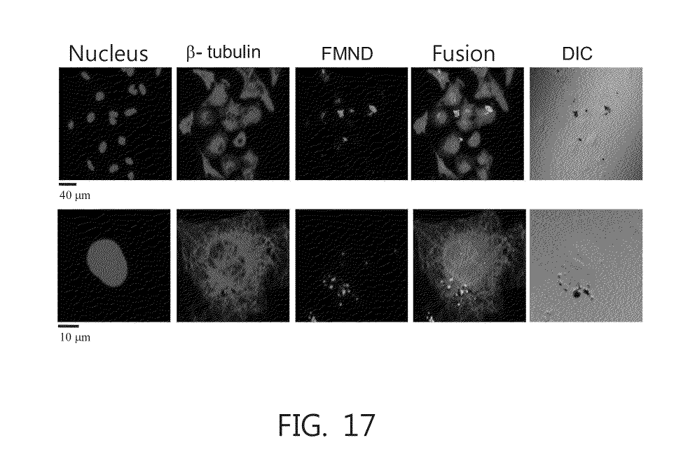

A549 cells were treated with or without 50 μg/ml of FMND for 24 hours. The FMND-bearing cells were separated by the magnetic device as described previously. The cell morphology, viability and growth ability of the FMND-bearing cells of different generations were similar to the parental cells (FIG. 14). In addition, the protein expression patterns of the FMND-bearing cells on SDS-PAGE were not altered (FIG. 15). Moreover, the FMND-bearing cells still carried the fluorescence intensities of FMNDs, and that these florescence intensities could be detected by the flow cytometer (FIG. 16) and the confocal microscope (FIG. 17).

### Cryopreservation, Re-Dissolution and Analysis of the Generations

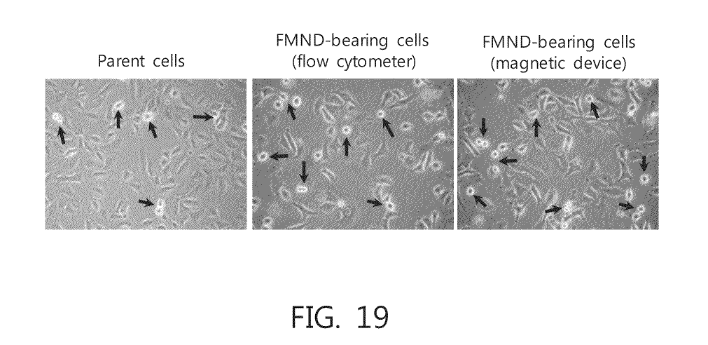

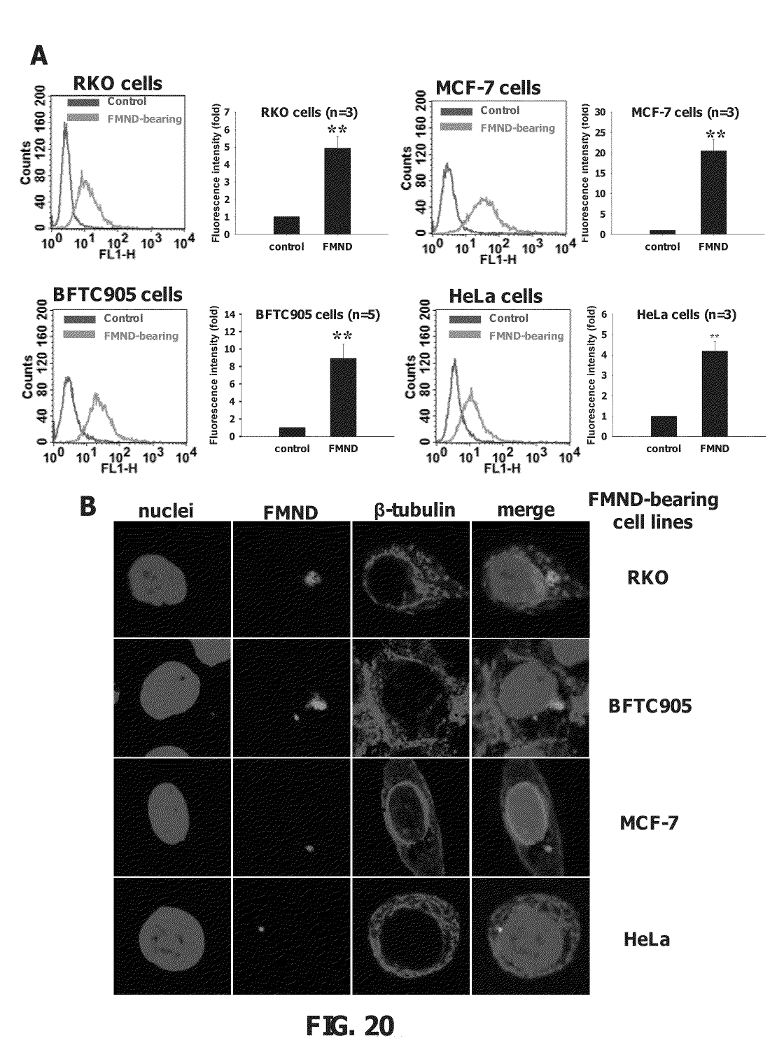

The separating ratio of the generations of the FMND-bearing cells was calculated by dividing the separated FMND-bearing cell number by the total cell number counted before separation. The separating ratio of the first generation had an average of 75.84%, and the separating ratio in the fourth generation had an average of 60%. The first generation of the FMND-bearing cell lines was stored in liquid nitrogen. After re-thawing of the FMND-bearing cells, the cell morphology and viability were still similar to the parental cells (FIG. 19). The arrows in FIG. 19 indicate round cells undergoing cell division. A variety of cancer cell types have been examined to illustrate the universal use of FMND-bearing cells. Human cancer cell lines including human colon (RKO), bladder (BFTC905), breast (MCF-7), and cervical (HeLa) were incubated with FMND particles (50 μg/ml) for 24 h, and then the FMND-bearing cells were separated by a magnetic device. The fluorescence intensities of FMNDs in the various cancer cell types were examined by flow cytometer. All cell lines had increased fluorescence intensities following treatment with FMNDs (FIG. 20). Quantified data showed that the fluorescence intensities of FMNDs were significantly increased in all cell lines (FIG. 20). The FMND-bearing cancer cell lines were confirmed by confocal microscopy; confocal microscopy showed that these FMND-bearing cancer cell lines retained the FMND particles, and exhibited green fluorescence (FIG. 20).

According to the present invention, a method for labeling cells using fluorescent magnetic nanodiamonds is provided. This invention also allows the use of the flourescent and magnetic properties to separate the cells which had been pre-labeled. According to the method of the present invention, the labeled or separated cells have cell morphology, viability and growth ability similar to those of the parent cells after sub-culturing or Cryopreservation. The method provided by the present invention is useful in the standardization, detection imaging or tracking of cells, the analysis of biomolecular activity and screening of drugs in the biomedical field.

The above examples only exemplify the principles and the effects of the present invention. They are not used to limit this invention. It is possible for those skilled in the art to modify and/or alter the above examples (whilst/before) carrying out this invention without contravening its spirit and scope. Therefore, the protection scope of this invention should be indicated as stated by the following claims.

What is claimed is:

1. A method for separating labeled cells, comprising:
culturing target cells;
co-culturing the target cells with a solution of flourescent magnetic nanodiamonds, to obtain a co-culture of fluorescent magnetic nanodiamonds and target cells, so as to allow the fluorescent magnetic nanodiamonds in the solution to label the target cells, to obtain fluorescent magnetic nanodiamond-labeled target cells; and
separating the fluorescent magnetic nanodiamond-labeled target cells from the co-culture of fluorescent magnetic nanodiamonds and target cells by fluorescent and magnetic properties of the fluorescent magnetic nanodiamond-labeled target cells.

2. The method of claim 1, wherein the fluorescent magnetic nanodiamond-labeled target cells have fluorescence intensity different from that of unlabeled cells, and the fluorescent magnetic-labeled target cells are sorted by using a flow cytometer, to obtain the sorted fluorescent magnetic nanodiamond-labeled target cells.

3. The method of claim 2, wherein the sorted fluorescent magnetic nanodiamond-labeled target cells are collected by eppendorfs with tube walls coated with PBS and containing complete media therein.

4. The method of claim 3, further comprising the step of cryopreserving the sorted fluorescent magnetic nanodiamond-labeled target cells.

5. The method of claim 1, wherein the fluorescent magnetic nanodiamond-labeled target cells are separated by using a magnetic device.

6. The method of claim 5, further comprising the step of suspending the fluorescent magnetic nanodiamond-labeled target cells and the unlabeled cells in a container with a buffer solution prior to using the magnetic device, so as to allow the magnetic device to facilitate the fluorescent magnetic nanodiamond-labeled target cells to be adhered onto the tube walls of the container.

7. The method of claim 6, further comprising the step of cryopreserving the sorted fluorescent magnetic nanodiamond-labeled target cells.

8. A separated cell, which is a cell separated by the method of claim 1.

9. The separated cell of claim 8, wherein the cell is an animal cell.

10. The separated cell of claim 9, wherein the animal cell is one of a cancer cell or a stem cell.

11. The separated cell of claim 10, wherein the cancer cell is a lung, colon, bladder, breast, or cervical cancer cell.

12. A use of the separated cell of claim 10 for standardization, detection, imaging or tracking of a cell.

13. A use of the separated cell of claim 10 for analysis of the activity of biomolecules and screening of drug activity.
